(19) United States
(12) Patent Application Publication
(10) Pub. No.: US 2012/0090627 A1
(43) Pub. Date: Apr. 19, 2012

(54) Dislodgement detector for intravascular implantable medical device

(76) Inventors: Terrence Ransbury, Chapel Hill, NC (US); Brad Pedersen, Minneapolis, MN (US)

(21) Appl. No.: 13/087,373

(22) Filed: Apr. 14, 2011

Related U.S. Application Data
(60) Provisional application No. 61/324,158, filed on Apr. 14, 2010.

(51) Int. Cl. A61B 19/00

(52) U.S. Cl. 128/899

(57) ABSTRACT

Systems, methods, and devices providing for improved safety for an Intravascular Implantable Device (IID) are described herein. The IID includes a dislodgement detector that may include at least one accelerometer device and/or one or more microphones. In various embodiments, the accelerometer device is adapted to sense various forces or motions such that a dislodgement event may be detected. In one embodiment, the detector is adapted to sense movement of the IID through the vascular system as a result of dislodgement. In another embodiment, the detector is adapted to detect and compare accelerations caused by the force of gravity to determine a dislodgement event. In various embodiments, the detector includes a multi-axis accelerometer. In one such embodiment, the detector is adapted to detect an orientation of the IID in order to determine a dislodgement event.

This application claims the benefit of U.S. Provisional Application No. 61/324,158, filed Apr. 14, 2010, which is incorporated herein by reference.

### FIELD OF THE INVENTION

The present disclosure relates generally to implantable medical devices. More particularly, the embodiments of the present disclosure relate to systems, methods and devices for detecting an intravascular implantable medical device dislodgement event.

### BACKGROUND

An implantable medical device (IMD) is an apparatus that is typically placed inside a living body to monitor certain physiological signals and provide therapy to an organ or tissue. A conventional IMD, such as a pacemaker, defibrillator or neurostimulator, is implanted subcutaneously in a convenient location beneath a patient's skin. Components of the IMD, such as electrical circuitry or batteries, are contained within a hermetically sealed housing. This housing is typically constructed to isolate IMD components within the housing from the human body. A typical IMD may include electrodes that are adapted to sense physiological conditions or to deliver therapy, for example the delivery of electrical energy to one or more portions of the heart of a patient. The IMD may include one or more leads that couple one or more electrodes to electrical circuitry disposed within the housing. An IMD may also include electrodes on one or more surfaces of the IMD housing.

In embodiments of an IID that incorporates one or more leads, the leads are typically adapted to carry current from the IMD to bodily tissue to stimulate the tissue in one of several ways depending upon the particular therapy being delivered. Leads may also be used to allow an IMD to communicate with one or more electrodes for sensing physiologic signals to determine when to deliver a therapeutic pulse to the tissue, and the nature of the pulse, e.g., a pacing pulse or a defibrillation shock. One or more catheter leads may be connected to an IMD to deliver drugs to various body parts for pain relief, defibrillation threshold reduction, and so forth.

Recently, intravascular implantable devices (IIDs) have been developed that are adapted to be implanted in the vascular system of a patient in contrast to the subcutaneous implantation of conventional IMDs. These elongated IIDs may take the form of a plurality of independent, substantially cylindrical or frustro-cylindrical housings, such as disclosed by U.S. Pat. No. 7,363,082 to Ransbury et al, U.S. Pat. No. 7,529,589 to Williams et al, and U.S. Pat. No. 7,840,282 to Williams et al, each of which is incorporated herein by reference as to features of the IID's. These housings may be connected together through a series of flexible components such as bellows so that the elongated implantable medical device is flexible enough to be introduced through and anchored within a vascular system of a patient.

Chronically anchoring an IID within the vasculature of a patient presents significant challenges to IID designers. Not only must the device withstand the turbulent environment in which it is disposed, it must also minimally interfere with patient health. In the unlikely event that an IID were to become dislodged there may be serious implications for a patient. For example, defibrillation and/or pacing efficiency may be reduced due to the change in position with respect to the heart. The IID could even disrupt, damage, or even puncture a blood vessel or internal organ.

As such, many solutions have been proposed for chronically anchoring an IID within the valculature of a patient to ensure that the IID does not become dislodged. One example of an anchor is similar to a conventional stent which is expandable upon introduction into a patient's vasculature. When expanded, the anchor is adapted to “sandwich” the IID between the anchor and the wall of the vessel. Various other solutions for chronically anchoring an IID in a vessel are described in U.S. Pat. No. 7,082,336 to Ransbury et al.

Known IID anchors are designed to ensure that an IID remains secured at a desired position within a human vasculature such that IID dislodgement as described above is very unlikely. However, due to the potential for complications that may result from a dislodgment event, IID anchoring is critical. As such, a need exists for improvements in IIDs to minimize any damage that may occur should an IID become dislodged from an anchored position.

### SUMMARY OF THE INVENTION

In various embodiments of the present invention, an IID incorporates a dislodgement detector adapted to provide a reliable indication that an IID has become dislodged from an initial anchoring position in the vascular system.

In some embodiments discussed herein, an IID dislodgement detector includes at least one separation detection circuit. The at least one separation detection circuit may be adapted to provide an electrical, optical, magnetic, or other coupling between an IID main body and an anchor adapted to secure the IID main body in the vascular system of a patient. In various embodiments, the separation detection circuit is arranged such that if IID main body becomes separated from the anchor, a detectable coupling between the main body and the anchor is severed, thus providing an indication of an IID dislodgement event.

In one embodiment, the separation detection circuit is an electrical conductor constructed to form a current loop between the main body and the anchor. According to this embodiment, the electrical conductor may be constructed and arranged such that the conductor will break should the main body become separated from the anchor. Also according to this embodiment, electrical circuitry disposed within IID main body may be adapted to cause a current to flow through the electrical conductor, and to monitor current flow through the electrical conductor. Should current cease to flow, the electrical circuitry may be adapted to provide an indication of an IID dislodgement event.

In some embodiments, an IID dislodgement detector as discussed herein may include one or more accelerometers adapted to detect forces and/or movement. In one such embodiment, the detector may be adapted to measure one or more g-vectors, or accelerations due to the force of gravity. In an embodiment, the detector is adapted to detect a magnitude of g-vector forces along a vertical axis. According to this embodiment, a comparison may be made with an established baseline g-vector amplitude (for example when the patient is in an upright, resting position) and a current g-vector amplitude to determine a dislodgement event.

In an embodiment, the detector may employ a multi-axis accelerometer. In one such embodiment, the detector may be adapted to detect g-vector accelerations along a plurality of dimensional axes, such as the X, Y, and Z axes. The detector may be adapted to determine, based on which axis accelerations due to the force of gravity are measured, an orientation of the detector. Due to the non-uniform orientation of vessels within a vascular system, detection of an orientation of the detector may provide an indication of IID position and thus an indication of an IID dislodgement event.

In one embodiment, the detector may be adapted to detect movements of the IID itself through a vascular system. According to this embodiment, the detector may monitor for accelerations indicating movement through the vascular system. Significant movement of art IID may indicate an IID dislodgement event.

In an embodiment, the detector may incorporate an accelerometer and/or microphone adapted to detect one or more forces and/or sounds emanating from cardiac function, such as the contraction and expansion of the heart. According to these embodiments, an amplitude of forces and/or sounds emanating from cardiac function may be utilized to determine a proximity of the detector to the heart. Monitoring of such forces and/or sounds may provide an indication of IID positioning and, as such, an indication of an IID dislodgement event.

In another embodiment, the detector may be adapted to determine whether or not a previously anchored portion of an IID is freely moving in the vascular system. The detector may be adapted to detect motion of an unexpectedly unanchored portion of the IID in directions perpendicular to an orientation of the vascular system at a position in which the detector is currently disposed.

In an embodiment, the detector may be adapted to be operated to adjust a frequency of testing at least partly determined by a relative risk of dislodgment. In one such embodiment, the detector is adapted to determine whether an initial time period after implantation has passed. According to this embodiment, the detector is operated to test for an IID dislodgement event relatively frequently for an initial time period after implantation, and relatively infrequently once the time period has passed. In other embodiments, the detector my be operated to test for an IID dislodgement event more frequently when a patient is in a state of high activity. According to these embodiments, one or more accelerometers included in the detector may be adapted to detect a patient's activity level, and adjust a frequency of dislodgement event testing based on the detected activity level. In one embodiment, the detector is operated to test for a dislodgement event for a time period following a high activity state of a patient.

In some embodiments, accelerometers may be located in a portion of the IID that is anchored or fixed, so as to minimize erroneous readings.

In use, the dislodgement detector may be used not only to detect anchor failure and substantial movements of IID (dangerous to patient), but also smaller scale shifts of the IID position that, while not necessarily dangerous to the health of the patient, may affect the proper function of the device. For example, for an implantable pacemaker, neurostimulator or defibrillator, a small shift in the device position can impact the ability of the stimulus from the device's electrodes to have the desired effect, or impact the function of sensors such as sensing electrodes.

### BRIEF DESCRIPTION OF THE FIGURES

The invention may be more completely understood in consideration of the following detailed description of various embodiments of the invention in connection with the accompanying drawings, in which:

FIG. 1 illustrates generally one example of an intravascular implantable device (IID).

FIG. 2 illustrates generally one example of an IID that includes an anchor and a IID dislodgement detector disposed within a vascular system of a patient.

FIGS. 3A and 3B illustrate generally examples of an IID dislodged from all initial anchoring position.

FIGS. 4A and 4B illustrate generally one example of an IID and an anchor disposed within a patient's vascular system.

FIGS. 5A-5C illustrate generally various embodiments of an IID that includes a separation detection circuit.

FIG. 6 is a flow chart that illustrates generally one embodiment of a method of detecting an IID dislodgement event.

FIGS. 7A-7C illustrate generally various embodiments of IIDs that include a dislodgment detector.

FIGS. 7D-F illustrate generally embodiments of a dislodgment detector of an IID.

FIGS. 8A-8F illustrate generally various embodiments of an IID dislodgement detector adapted to detect accelerations caused by the force of gravity to determine a dislodgement event.

FIGS. 9A-9F illustrate generally various embodiments of an IID dislodgement detector adapted to detect the proximity of the detector to the heart to determine a dislodgement event.

FIGS. 10A-10B illustrate generally one embodiment of an IID detector adapted to determine whether a previously anchored portion of an IID is freely moving to determine a dislodgement event.

FIG. 11 illustrates generally one embodiment of a method of determining an IID dislodgement event.

FIG. 12 illustrates generally one embodiment of a method of operating an IID dislodgement detector to test for a dislodgement event based on one or more indications of high risk conditions.

FIG. 13 illustrates generally one embodiment of a method of operating an IID dislodgement detector to test for a dislodgement event at a higher frequency for an initial time period after the IID is anchored in a vascular system.

FIG. 14 illustrates generally one embodiment of a method of operating an IID dislodgement detector to test for a dislodgement event at a higher frequency based one or more indications that the patient is in a state of high activity.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE PREFERRED EMBODIMENTS

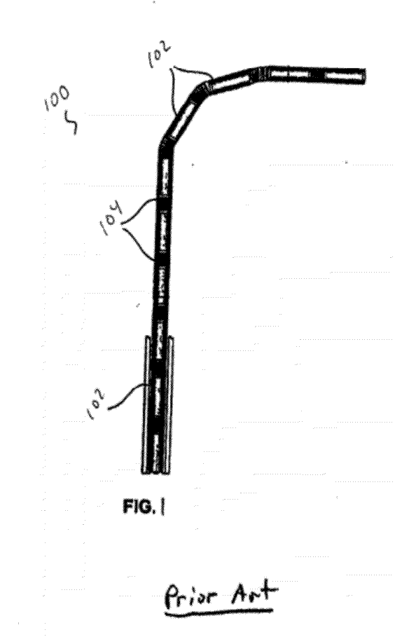

FIG. 1 and the below descriptions of FIG. 1 are presented to explain the nature of recent developments providing for implantable intravascular devices (IIDs) that may be chronically implanted in the vasculature of a patient. One of skill in the art will recognize that the invention described herein is not limited to the IIDs discussed in FIG. 1. Furthermore, one of skill in the art will recognize that the instant invention is applicable to any medical device adapted to be disposed in a fluidic in-patient environment.

In the illustrated embodiment, IID 100 includes a plurality of rigid or semi-rigid housings 102 that are constructed to enclose one or more IID components, such as electrical circuitry or batteries, to isolate them from a vascular environment. IID 100 may further include bendable portions 104 that link housings 102 together in such a way that IID 100 is flexible enough to be introduced to and chronically disposed in a vascular vessel of a patient. Bendable portions 104 may be a bellows, as depicted, or any other readily bendable components.

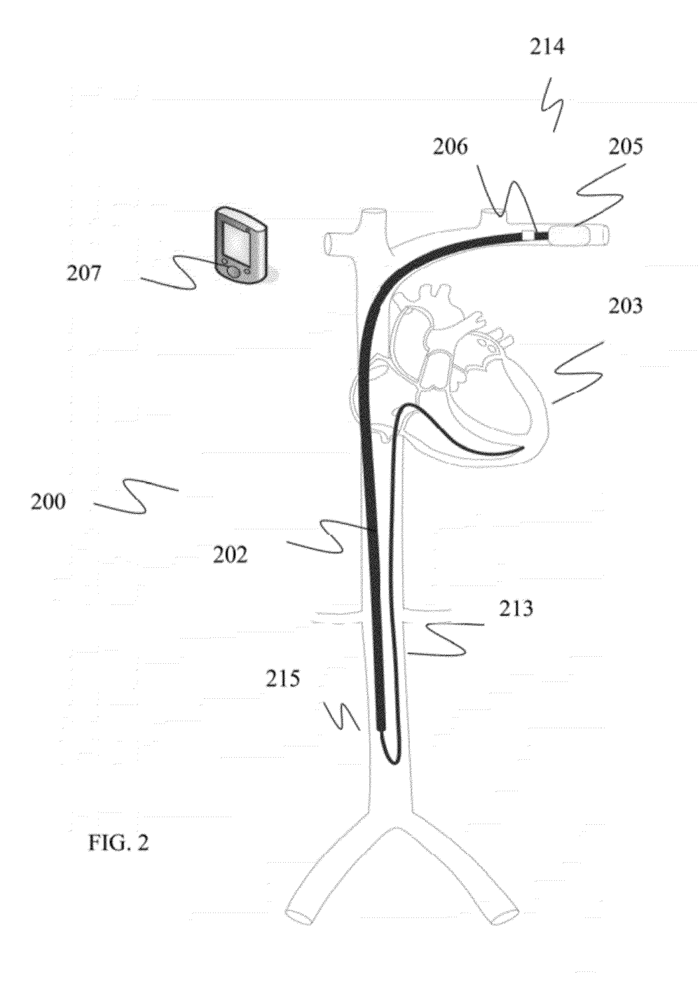

FIG. 2 illustrates one embodiment of an IID 200 that has been introduced to and disposed in a patient's vascular vessel 201 according to various aspects of the invention described herein. In this particular embodiment, IID 200 has a first end 214 secured at a position in the subclavian vein by anchor 205. IID main body 202 extends through the subclavian vein, superior vena cava, and inferior vena cava and a second end 215 of main body 202 is left unanchored in the inferior vena cava. At second end 215, IID 200 includes one or more leads 213 adapted to be introduced to and disposed in various locations, such as heart 203 as depicted, to sense hemodynamic conditions and/or deliver therapy. Embodiments of IID 200 positioning discussed herein are provided for purposes of illustration only, and one of skill in the art will recognize that any positioning and/or anchoring configuration of IID 200 and leads 213 in a vascular system are within the spirit and scope of the invention described herein. Further description and examples of IID anchors can be found in U.S. Pat. Nos. 7,363,082 and 7,082,336 to Ransbury et al., which are hereby incorporated by reference in their entireties.

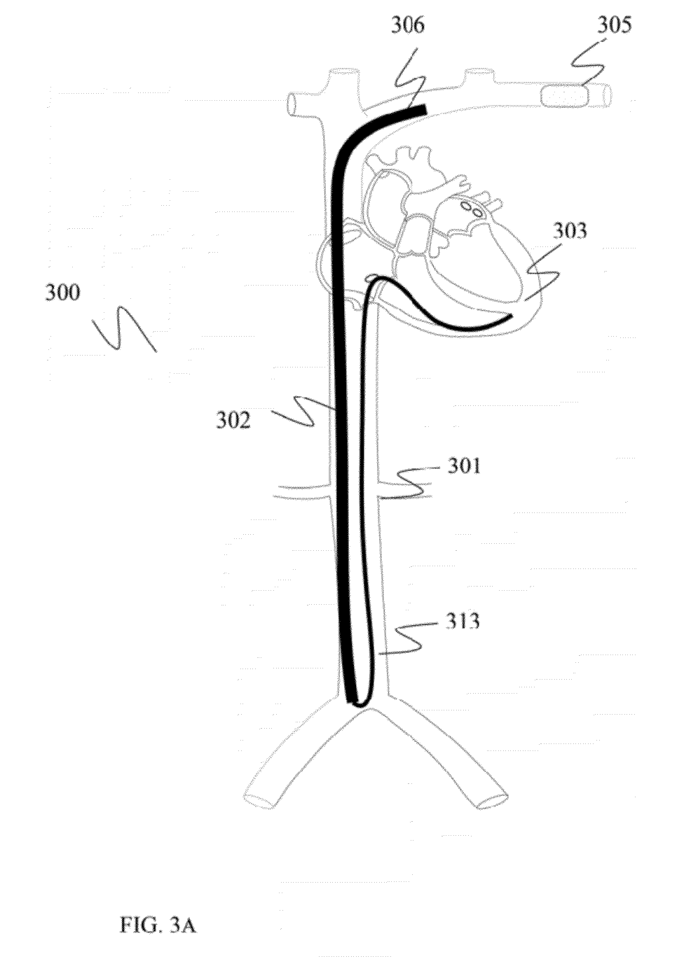

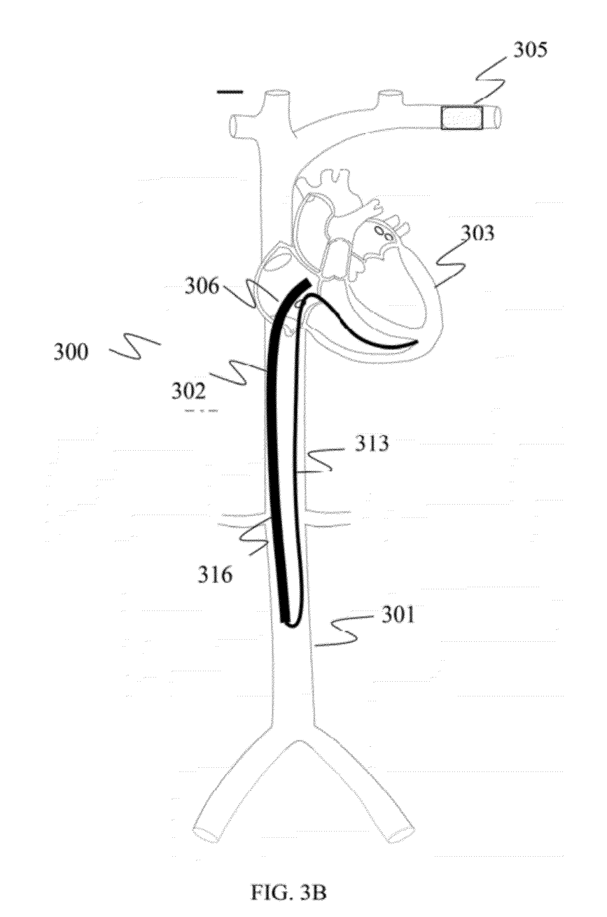

FIGS. 3A and 3B illustrate generally examples of IID 300 dislodgement events, the consequences of which embodiments of the invention described herein attempt to minimize or prevent. A dislodgment event occurs when a once anchored IID, such as IID 300, becomes displaced from a position at which it was initially secured. A dislodgement event may occur should IID main body 302 and anchor 305 become dislodged from an initial position. A dislodgement event may also occur when IID main body 302 becomes separated from anchor 305.

FIG. 3A, depicts a relatively longer IID 300 that has come loose from anchor 305, and has traveled a distance through vascular system 301, and FIG. 3B depicts a shorter IID 300 that has become dislodged, an upper portion of which is lodged in a chamber of heart 303. Should a dislodgement event such as those illustrated occur, damage may be caused to vascular tissue by IID 300 traveling uncontrolled through the vasculature. IID 300 may even become partially lodged in the heart and disrupt the hearts ability to circulate blood as illustrated in FIG. 3B. In a worst case scenario, the dislodgement of IID 300 from its original position could result in damage to or even puncturing of vascular or cardiac tissue.

Referring back to FIG. 2, in order to improve the ability of physicians or patients to react to the hazardous consequences discussed above, IID 200 includes dislodgement detector 206. Dislodgement detector 206 may be adapted to provide a reliable indication that an IID dislodgement event has occurred.

Also depicted in FIG. 2 is telemetry device 207. Telemetry device 207 may be any device capable of wirelessly communicating with IID 200, such as to deliver commands, exchange information, or to program IID 200. In various embodiments, circuitry of IID 200 itself may be adapted to control operation of detector 206, for example when detector 206 is operated to determine whether a dislodgement event has occurred, or to process signals detected by detector 206. In other embodiments, circuitry of IID 200, and detector 206, may be controlled and/or programmed via communication enabled by telemetry device 207. In one embodiment, telemetry device 207 is a battery powered device adapted to be carried by or on the patient for the period of time until IID 200 is secured within the vasculature by virtue of fibrosis.

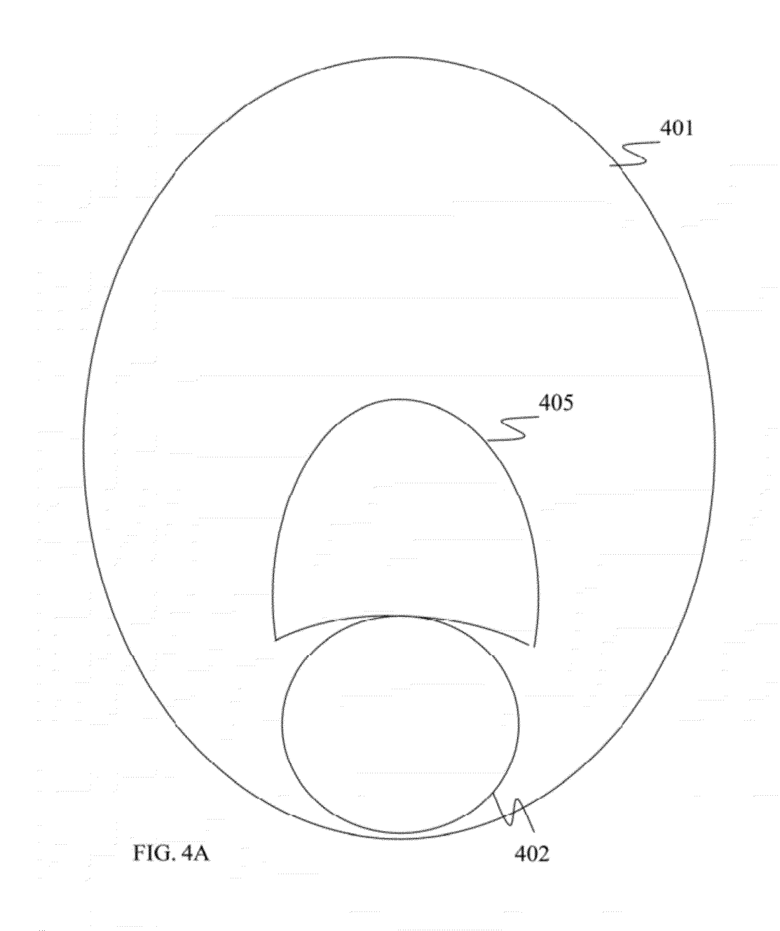

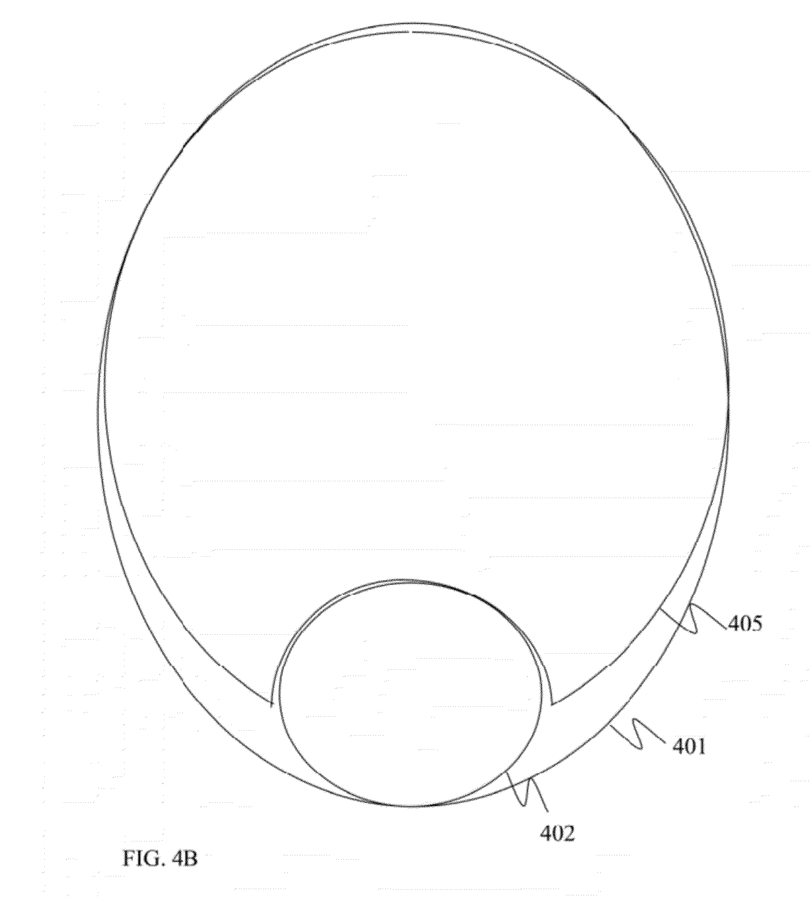

FIGS. 4A and 4B illustrate generally cross-sectional views of one example of an IID anchor 405 disposed within a vessel 401 of a patient's vascular system. The anchor depicted is similar to a classical stent. Anchor 405 is constructed to maintain a collapsed position upon introduction into vessel 401. As shown in FIG. 4B, once IID 400 has been introduced and is in a desirable position, anchor 405 is expandable towards the walls of vessel 401. As such, anchor 405 is constructed to “sandwich” IID main body 402 against a wall of vessel 401 to secure a position of IID 400.

Typically, an anchored IID 400 is more likely to dislodge during a relatively short time period after IID 400 is first anchored in a patient's vascular system. This is in part due to the buildup of blood cells (sometimes referred to as fibrosis) that adhere to and collect on exposed surfaces of both the IID and anchor. This buildup may eventually assist the stability of the IID anchoring arrangement. Prior to substantial buildup, the IID may be more prone to a dislodgement event.

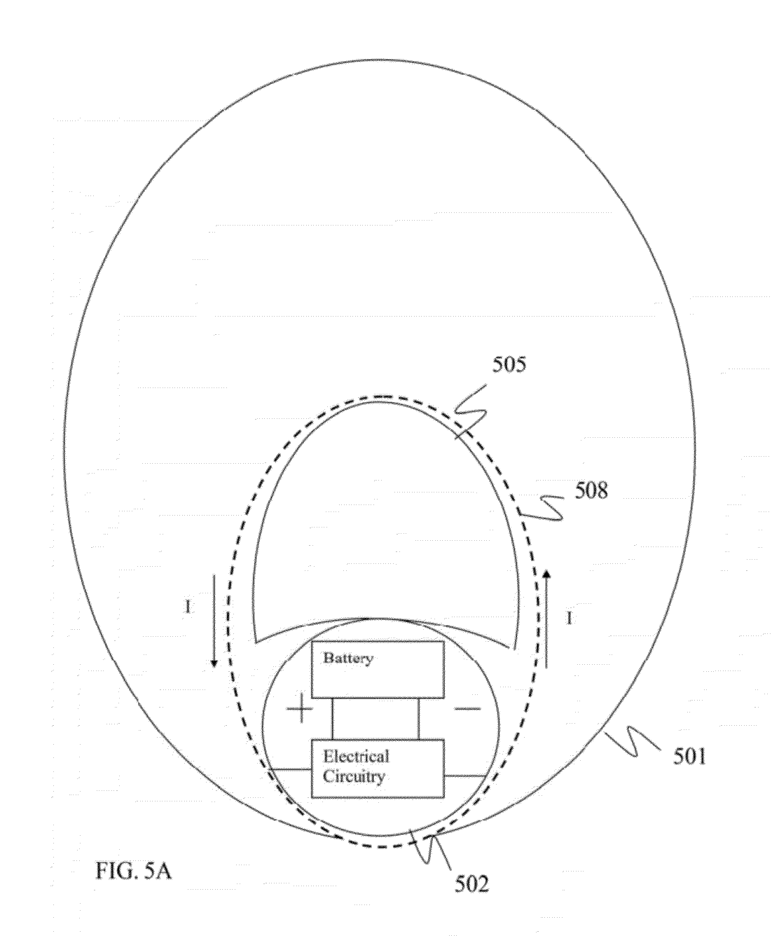

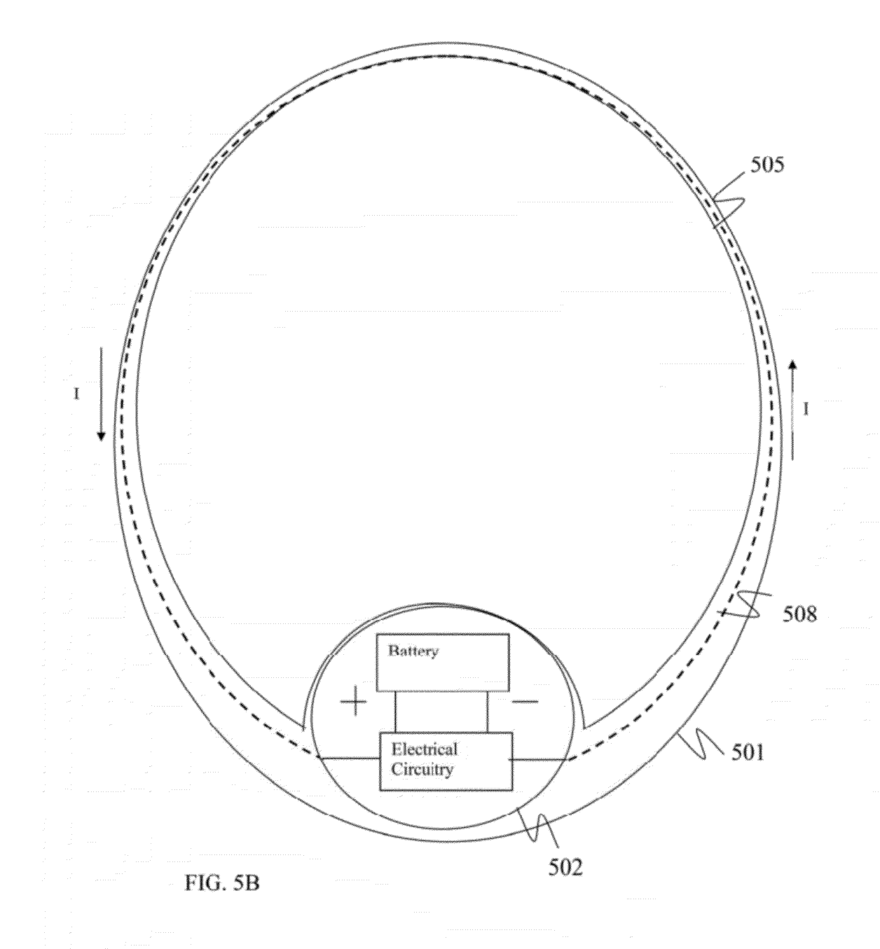

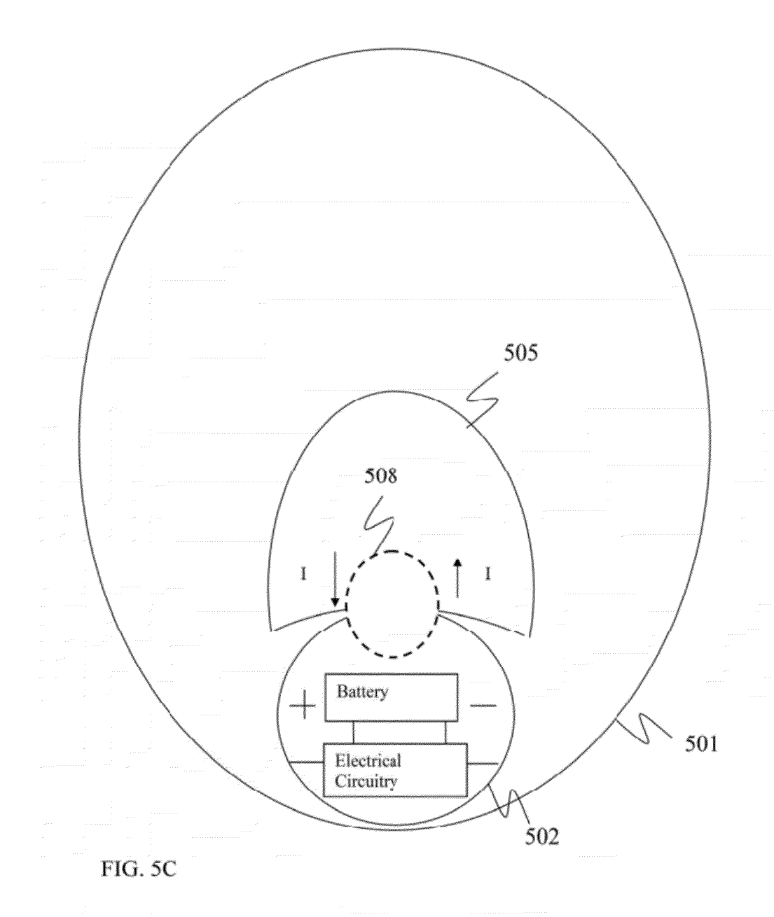

FIGS. 5A-5C illustrate generally an embodiment of an IID 500 that includes a separation detection dislodgement detector 508 according to various aspects of the invention described herein. FIG. 5A illustrates IID 500 with an anchor 505 in a non-extended position. Separation detection circuit 508 may be constructed to surround at least a portion of both IID main body 502 and anchor 505. In the FIG. 5A embodiment, separation detection circuit 508 is constructed to surround a periphery of both main body 502 and anchor 505. In the embodiment depicted in FIG. 5C, however, separation detection circuit 508 is constructed to surround only a portion of main body 502 and anchor 505. IID components disposed within main body 502 may be electrically coupled to separation detection circuit 508 and adapted to cause a current (I) to flow through separation detection circuit 508 and to detect whether or not current is flowing through the circuit. In an embodiment, separation detection circuit 508 is arranged such that an open circuit is created if main body 502 separates from anchor 505. In one embodiment, separation detection circuit 508 is an electrical conductor adapted to break, and thus create a detectable open-circuit, if main body 502 separates from anchor 505.

In other embodiments not depicted in FIGS. 5A-5B, separation detection circuit 508 may be adapted to determine a dislodgement event based on signals other than an electrical signal. In one such embodiment, separation detection circuit 508 comprises an optical separation detection circuit. According to this embodiment, main body 502 may include components adapted to project an optical signal that may be reflected by a portion of anchor 505, and the reflected signal may be detectable such that if main body 502 is separated from anchor 505 the optical signal is no longer reflected thus providing an indication of a dislodgement event. In another embodiment, main body 502 may be magnetically coupled to anchor 505. According to this embodiment, main body 502 and anchor 205 may each include at least one transformer winding, the windings arranged such that a detectable magnetic coupling exists when main body 502 is secured by anchor 505. In an embodiment, the windings are arranged such that if main body 502 becomes dislodged from anchor 505, the magnetic coupling ceases to exist, thus providing a detectable indication of a dislodgement event. In some embodiments, separation detection circuit 508 is arranged to run continuously. In other embodiments, separation detection circuit 508 is arranged to run only periodically. In some embodiments of the periodic embodiments, the periodicity of actuation of separation detection circuitry 508 is increased after the device is implanted until an expected date after which IID 500 will be additionally secured due to fibrosis. Thereafter the period of actuating separation detection circuit 508 is lengthened to conserve battery life.

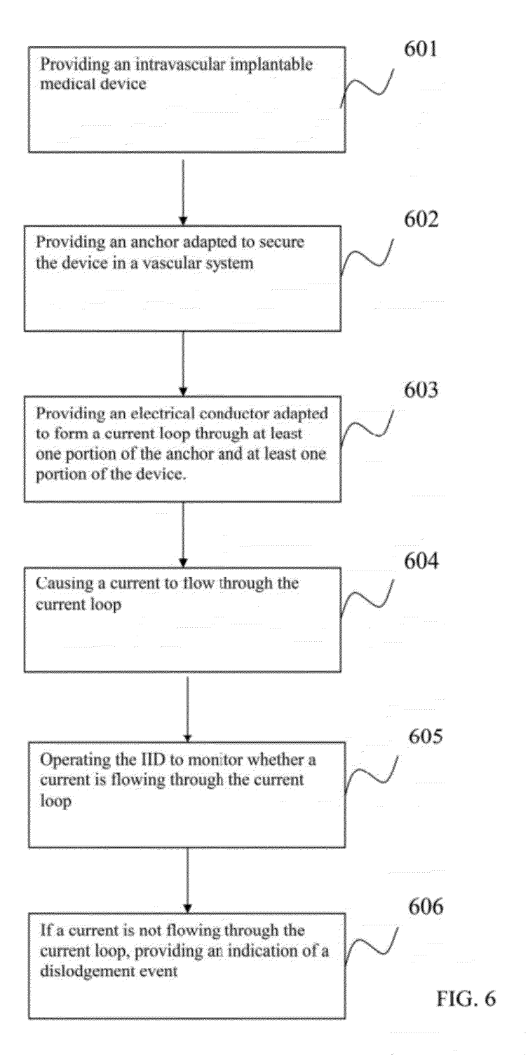

FIG. 6 illustrates generally a method of providing an IID dislodgement detector according to various aspects of the invention described here. At 601, an intravascular implantable medical device is provided. At 602, an anchor is provided for the IID that is adapted to secure the IID at a position within a vascular vessel of a patient. At 603, an electrical conductor is arranged to form a current loop between at least one portion of the anchor and at least one portion of the medical device. At 604, the IID is adapted to cause current to flow through the current loop. In an embodiment, separation of the device from the anchor causes a detectable break in the electrical conductor. At 604, the IID is adapted to monitor current flow through the current loop. At 605, the IID is adapted to detect that no current is flowing through the current loop. At 606, if the IID detects that no current is flowing through the current loop, a signal indicating a dislodgement event may be generated.

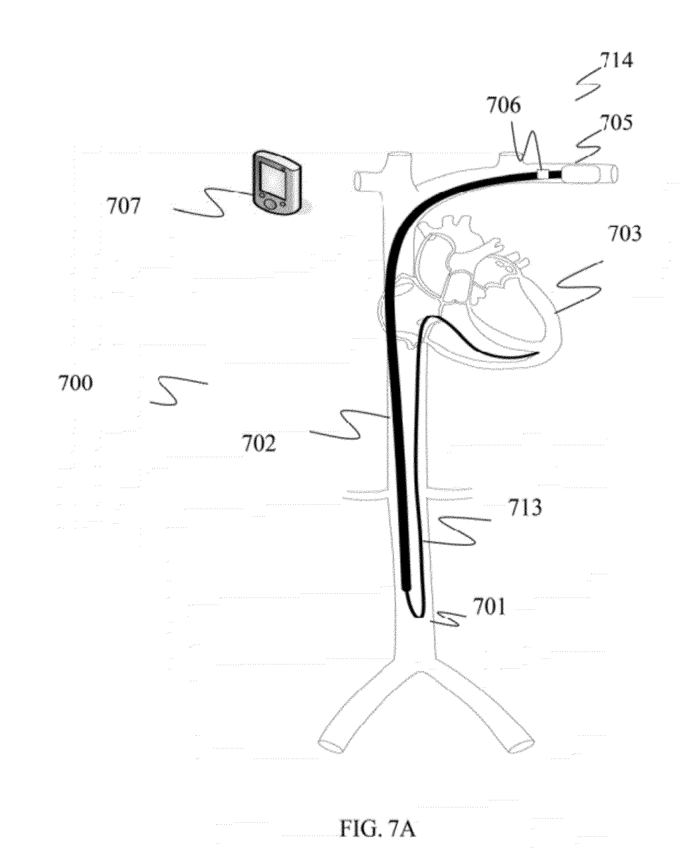

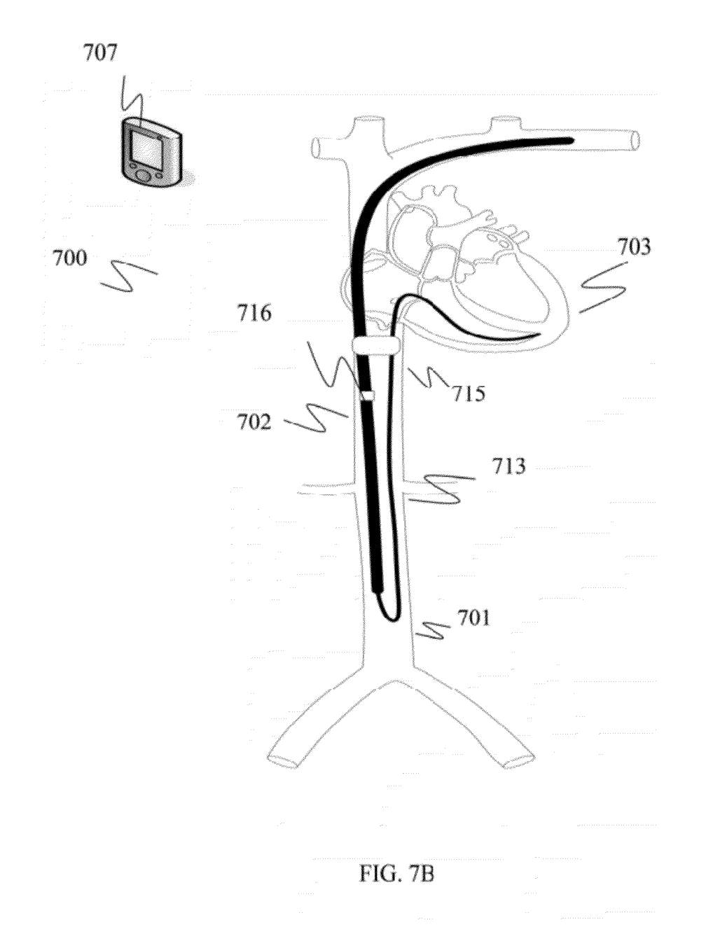

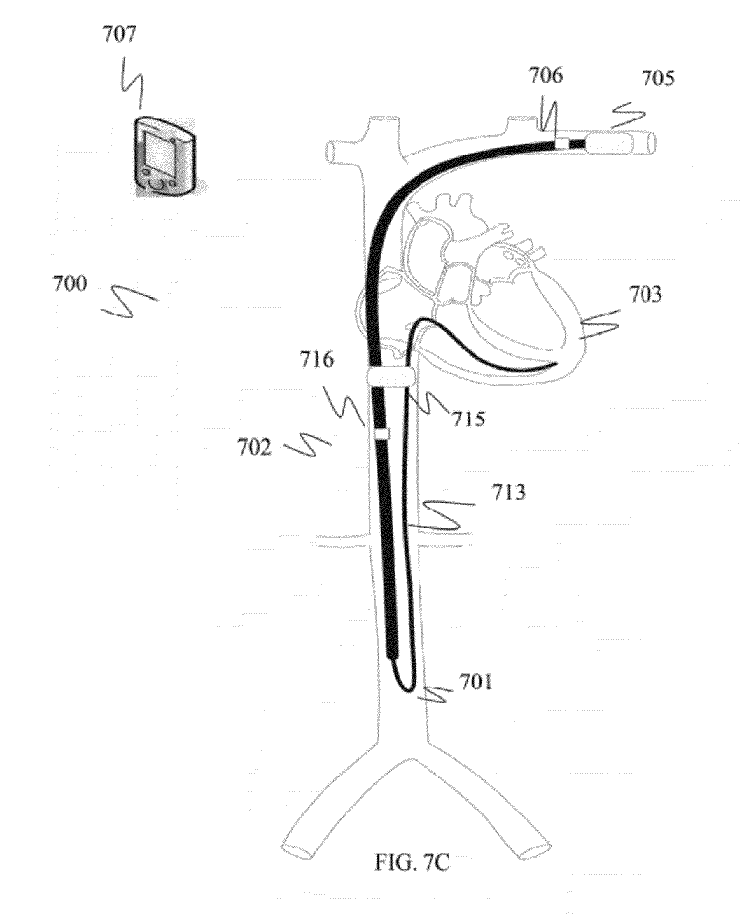

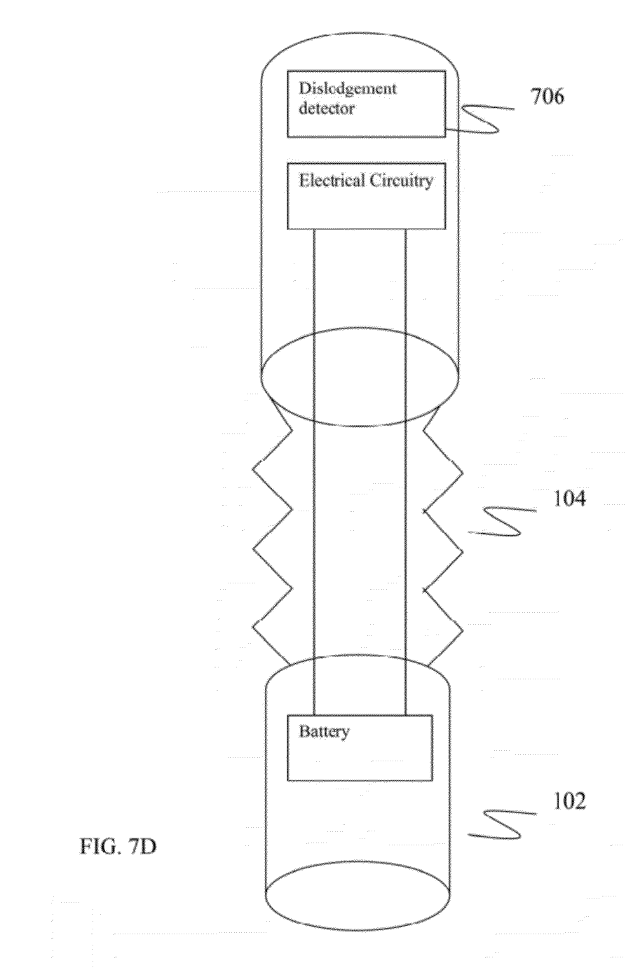

FIGS. 7A-E illustrate generally various embodiments of positioning and arrangement of anchor 705 and dislodgement detector 706 with respect to main body 702 of IID 700. According to these embodiments, dislodgement detector 706 does not include a separation detection circuit as described above with respect to FIGS. 5A-5C and FIG. 6. Instead, the embodiments discussed in below include a dislodgment detector that includes an accelerometer or microphone according to various aspects of the invention described herein.

FIG. 7A illustrates anchor 705 disposed at a top end 714 of IID main body 702 and dislodgement detector 706 arranged proximal to anchor 705. FIG. 7B illustrates anchor 715 disposed at a middle portion of main body 702 and dislodgement detector 716 arranged proximal to anchor 715. In alternative embodiments not depicted, dislodgement detector 706 or 716 may be disposed at a location distal to anchor 705 or 715. One of skill in the art will recognize that anchor 705 or 715 and dislodgement detector 706 or 716 may be disposed at any location along main body 702.

FIG. 7C illustrates an alternative embodiment where IID 700 includes first anchor 705 and second anchor 715, and first dislodgement detector 706 and second dislodgement detector 716. One of skill in the art will recognize that any number of dislodgement detectors, anchors, or combinations of these components arranged at any part of IID 700 are within the spirit and scope of the invention described herein.

FIGS. 7D-E illustrate generally various embodiments of arrangements of dislodgement detector according to various aspects of the invention described herein. In FIG. 7D, dislodgement detector 706 is shown disposed within an IID housing 102 as discussed above with respect to FIG. 1. As shown, dislodgement detector 706 is shown disposed in housing 102 along with electrical circuitry 720. Electrical circuitry 720 is coupled, via bendable portions 104, to battery 721 contained within second housing 102. One of skill in the art will recognize that dislodgement detector 706 may be disposed stand-alone within housing 102, or may be disposed along with IID components such as electrical circuitry 720 or battery 721. Electrical circuitry 720 may be adapted to monitor an output of dislodgement detector 706 to detect a dislodgement event. Electrical circuitry 720 may also be adapted to transmit information detected by dislodgement detector 706 to an external device such as telemetry device 707.

FIG. 7E illustrates an alternative embodiment wherein dislodgement detector 706 is not contained within a housing 102 of IID 700. Instead, dislodgement detector 706 includes an independent housing which is mechanically secured to at least one housing 102 and/or at least one bendable portion 104 of IID 700.

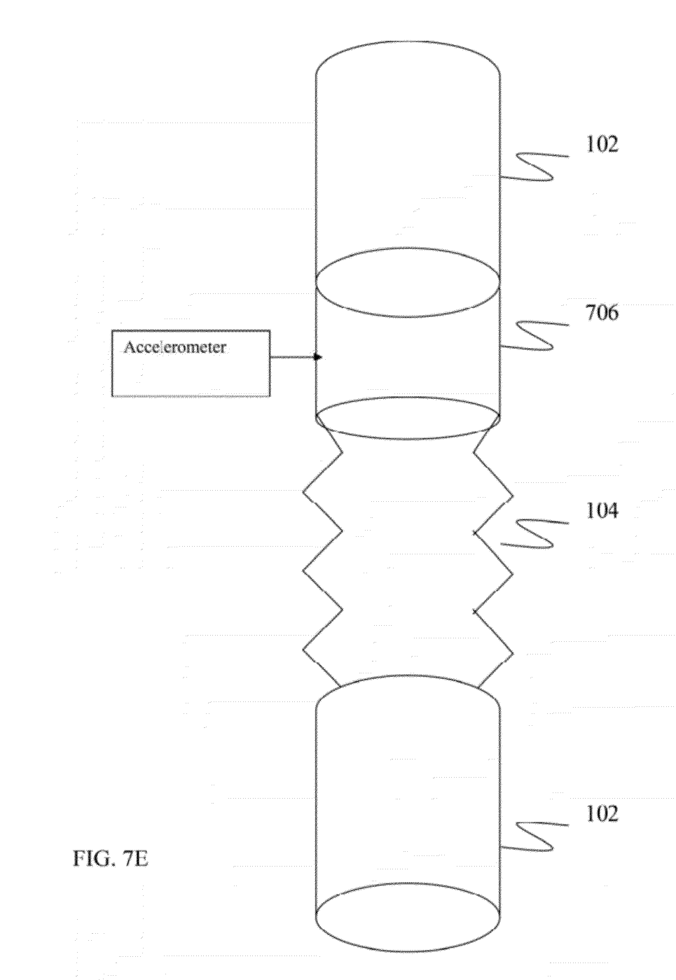

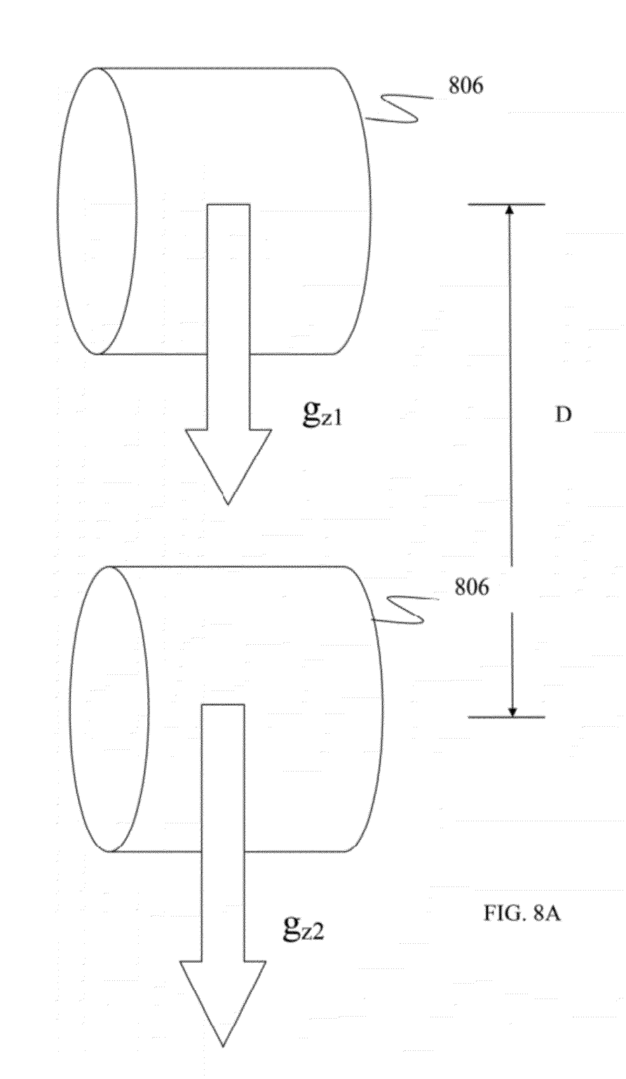

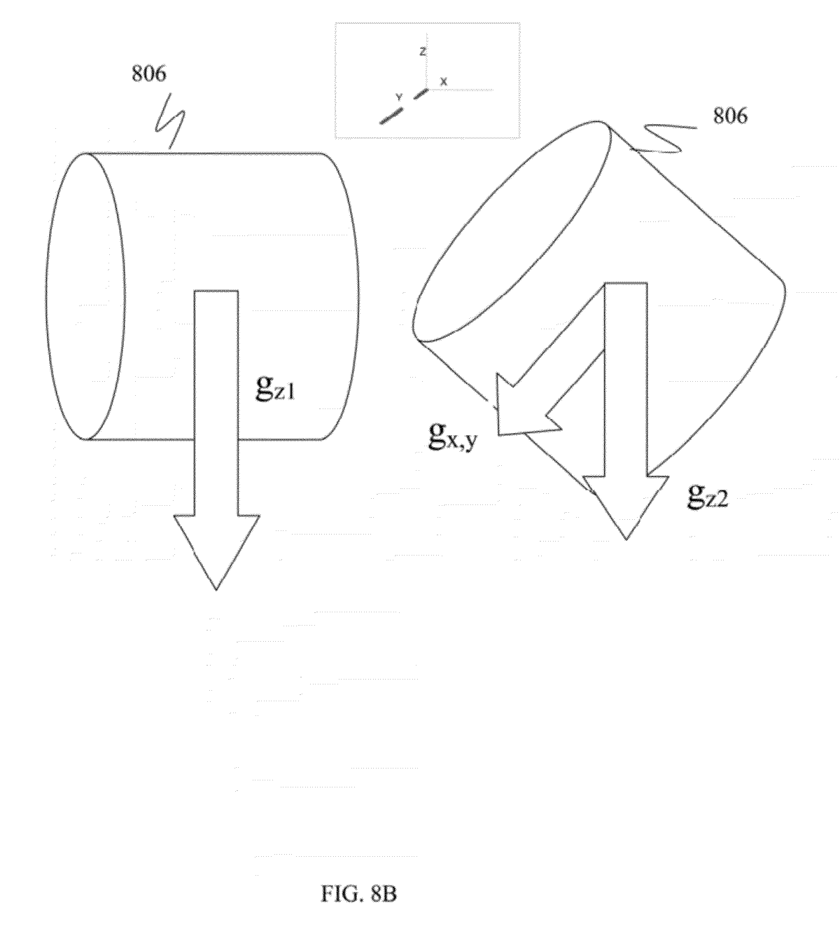

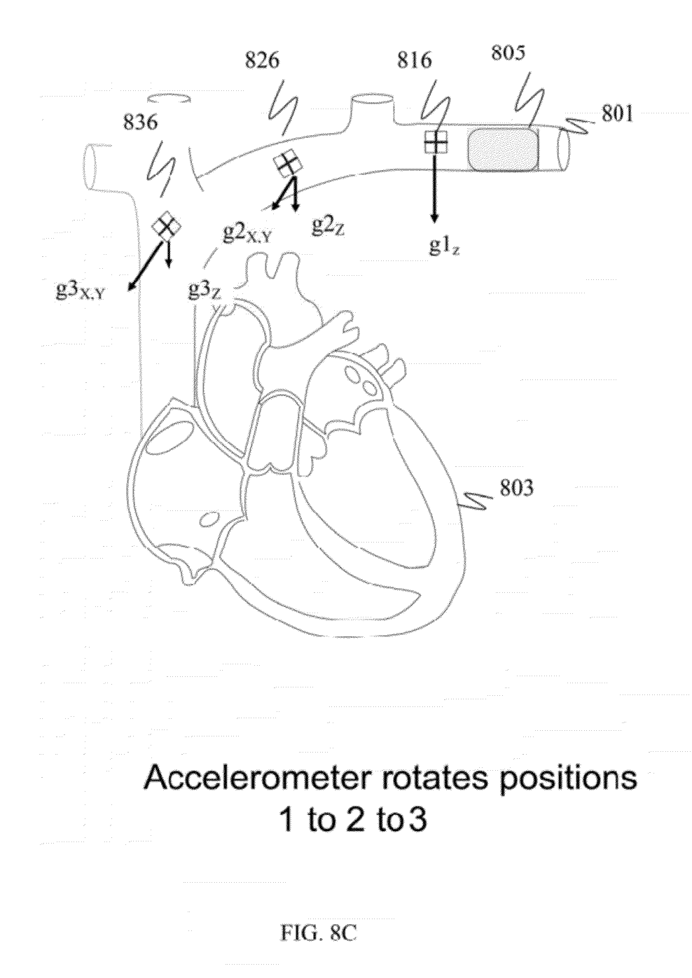

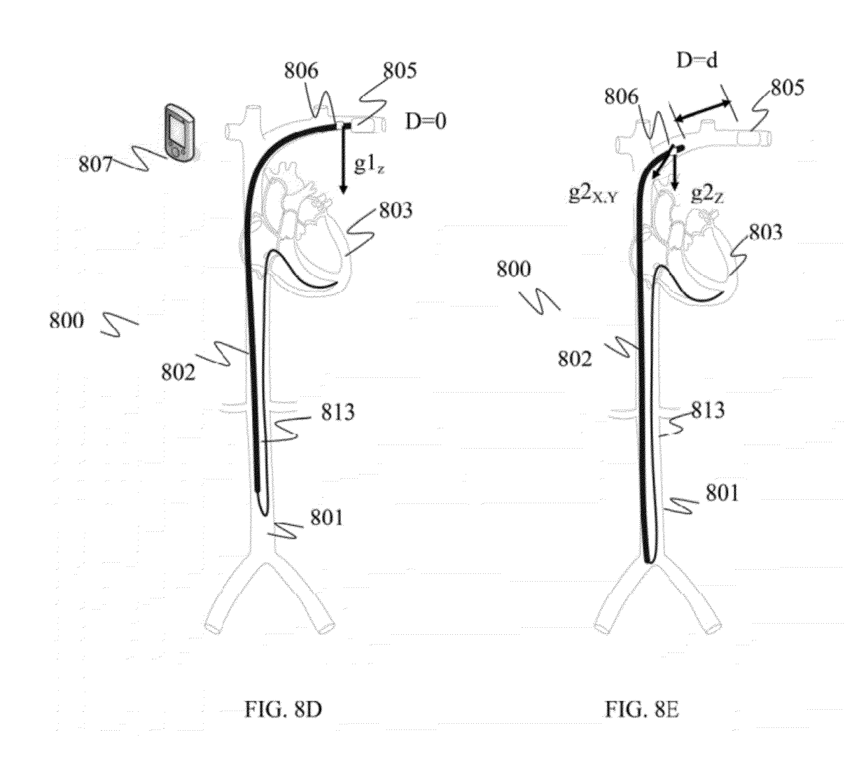

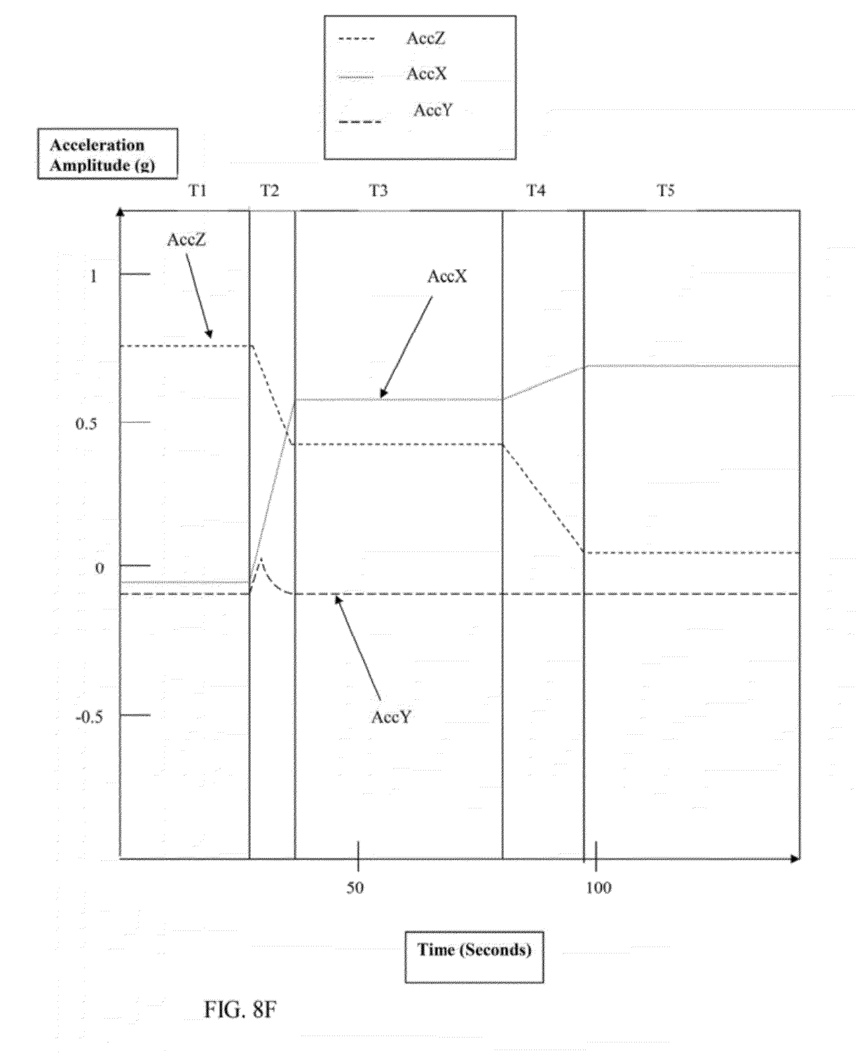

FIGS. 8A-8F illustrate generally various embodiments of accelerometer-based dislodgement detectors adapted to sense one or more indications of force or motion in order to determine an IID dislodgement event according to various aspects of the invention described herein. An accelerometer as discussed herein is an electrical, mechanical, or electro-mechanical device that is well known in the art and used in a variety of applications to provide detection of force, velocity, or acceleration. One of skill in the art will recognize that any accelerometer or other device capable of detecting force and/or motion now known or later developed is within the spirit and scope of the embodiments described herein.

FIG. 8A illustrates one embodiment of an accelerometer-based IID dislodgement detector 806. Detector 806 includes one or more accelerometers. In various embodiments, the one or more accelerometers may be adapted to provide one or more indications of accelerations imparted on detector 806 by gravity. These indications may be referred to as g-vectors. G-vectors may indicate an amplitude and/or direction of accelerations due to gravity.

As shown in FIG. 8A, when detector is located at a first position in a vascular system it may detect a first gravity vector (g-vector) g_{z1} in the vertical direction, or along a Z-axis. When detector 806 has moved a distance D, a second gravity vector g_{z2} may be detected. Monitoring of the g-vector along the Z-axis may provide a detectable change in g-vectors due to the position of detector 806, and thus detector 806 may provide an indication of an IID dislodgement event.

FIGS. 8B-8E illustrate generally various embodiments of IID dislodgement detector 806 that include a multi-axis accelerometer. G-vector forces measured using a multi-axis accelerometer may include both an amplitude (the amount of acceleration caused by the force of gravity), and a direction (along what directional axis the force was detected). As shown in FIG. 8B, a multi-axis accelerometer may be adapted to measure forces or movement along two or more axes, such as the X, Y, and Z axes. As such, should the orientation of detector 806 change, the acceleration imparted on the accelerometer by the force of gravity may modify the amplitude, and/or on what axis, the acceleration is detected. According to embodiments including a multi-axis accelerometer, by detecting g-vector forces along a plurality of axes, an orientation of detector 806 may be detected and utilized to determine an IID dislodgement event.

Due to the non-uniform orientation of a typical vascular system, should an IID's position change, an orientation of dislodgement detector 806 disposed along the IID will also change. As a result of this change in orientation, g-vectors may change and provide an indication of a dislodgement event. For example, as shown in FIG. 8C, detector 806 is initially disposed at a location 816 in a substantially horizontal portion of vascular system 801. The acceleration imparted by gravity on detector 806 in this position may be primarily represented by g-vector g1_{z}, because little or no acceleration is detected along the X or Y axis. However, when detector 806 has moved to a second position 826 in vascular system 801 that is not substantially horizontal, the detected acceleration due to gravity may be represented by g-vectors along multiple axis, such as the X or Y axis in addition to the Z-axis. In position 836, detector 806 is oriented even further from the Z-axis, and as such, detected acceleration due to gravity may be represented by g-vector forces with an even a larger amplitude along the X or Y axes than at position 826. As such, an IID that incorporates detector 806 may compare g-vector amplitudes and directions in order to determine that an IID has changed positions and thus provide an indication of a dislodgement event.

FIG. 8D illustrates generally IID 800 that includes a multi-axis accelerometer disposed at a first position in vascular system and shows a first g-vector g1, detected primarily along the Z axis, while FIG. 8E shows IID 800 dislodged from anchor 805 and disposed at a second position, and accelerations due to the force of gravity represented by two or more g-vectors, g2_{z} and g2_{x,y}. Because detector 806 has a different orientation from the first position to the second position, detector 806 may be able to provide an indication of a dislodgement event by comparing g-vectors as described above.

FIG. 8F illustrates generally a graph depicting the results of an experiment performed by the inventors utilizing an IID 800 that includes a multi-axis accelerometer as discussed above implanted in an animal test subject. As shown in FIG. 8F at an initial time period T1, the IID was anchored at a desired position in the animal patient's vascular system. At time period T2, the device became dislodged and is moving in the vascular system. At time period T3, the device has come to rest at a second position in the vascular system. At time period T4, the device is again moving in the vascular system. At time period T5, the device has come to rest at a third position in the vascular system.

As depicted in FIG. 8F, the incorporation of an accelerometer-based dislodgement detector 806 as discussed herein allows for detection of forces and motion to determine a dislodgement event. In one embodiment, acceleration vectors caused by the force of gravity may be compared to one another, for example the acceleration vectors at time period T1 compared to time period T3 or T5 may indicate a change in orientation (such as what axis detects acceleration due to the force of gravity), and/or amplitude, of g-vectors. In another embodiment, detector 806 may be adapted to detect motion of the IID itself instead of accelerations caused by gravity. According to these embodiments, detector 806 may be adapted to monitor for various indications that the IID (via the accelerometer included in the IID) itself is moving. In an embodiment, detector 806 may be adapted to compare indications of IID movement to one or more predetermined thresholds to determine a dislodgement event.

In an embodiment, a baseline g-vector may be established when IID 800 is known to be desirably disposed a vascular vessel (for example an upright, resting position). According to this embodiment, a detected g-vector may be compared to one or more thresholds indicating the established baseline g-vector to determine whether IID dislodgement has occurred.

In some embodiments, dislodgement detector 806 as depicted in FIGS. 8A-F may be adapted to differentiate relevant detected g-vectors from other forces imparted on an accelerometer, such as forces caused by patient movement. Dislodgement detector 806 may be adapted to compare a detected g-vector to one or more thresholds in order to differentiate relevant g-vectors from other forces.

In some embodiments, dislodgement detector 806 may be disposed at a position proximal to anchor 805 to minimize movement of detector 806 to enable accurate detection (and/or differentiation) of g-vector forces.

In some embodiments, IID 800 includes a plurality of dislodgement detectors 806, or a plurality of accelerometers. According to these embodiments, an ability of dislodgement detectors 806 to determine a difference in detected g-vectors may be improved by enabling detection of a position change at multiple points along IID 800. The use of multiple dislodgement detectors 806 (or accelerometers) may also enable an improved ability to distinguish between relevant detected g-vector forces and other forces, including those caused by patient activity.

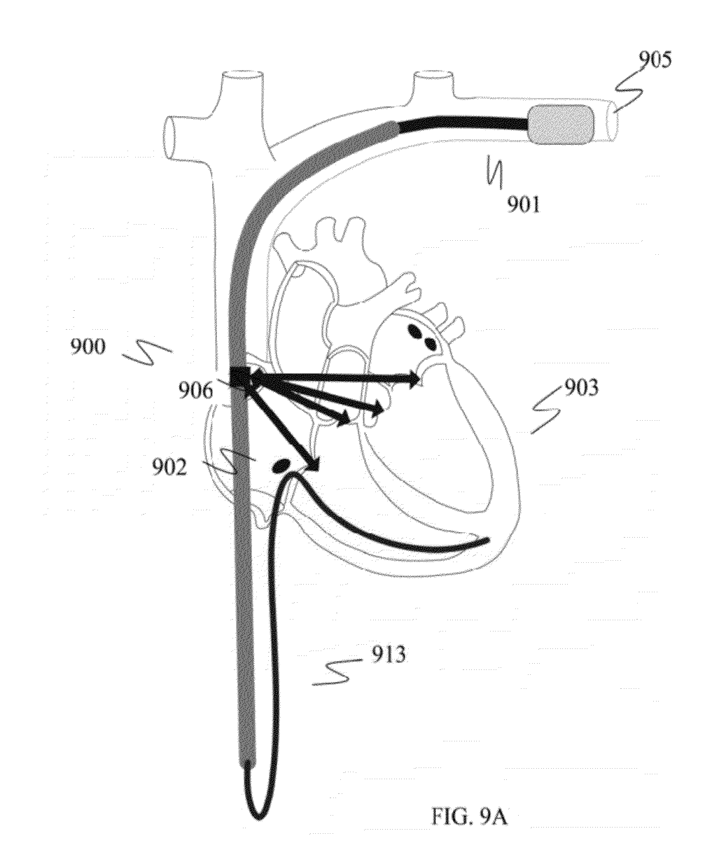

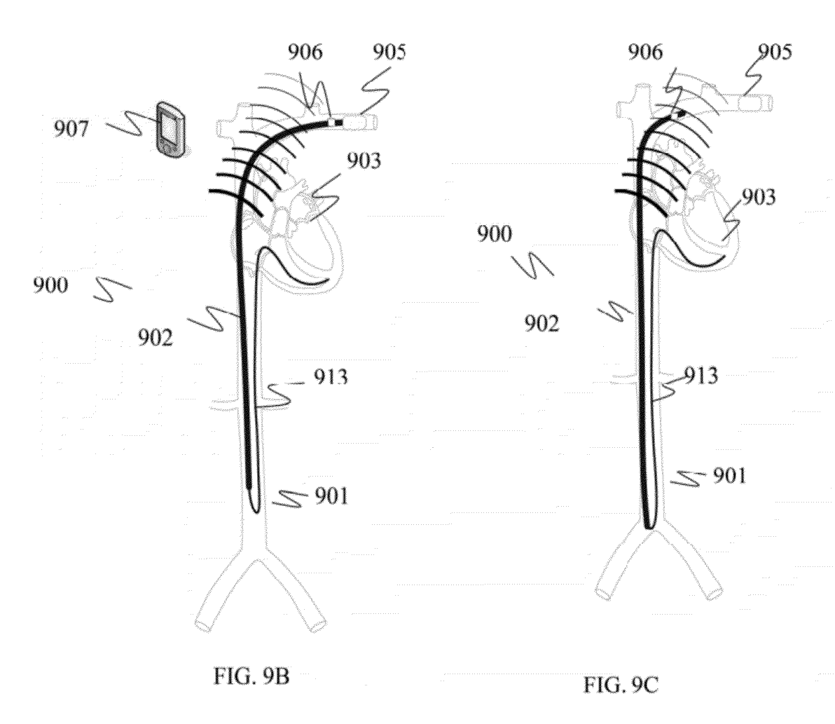

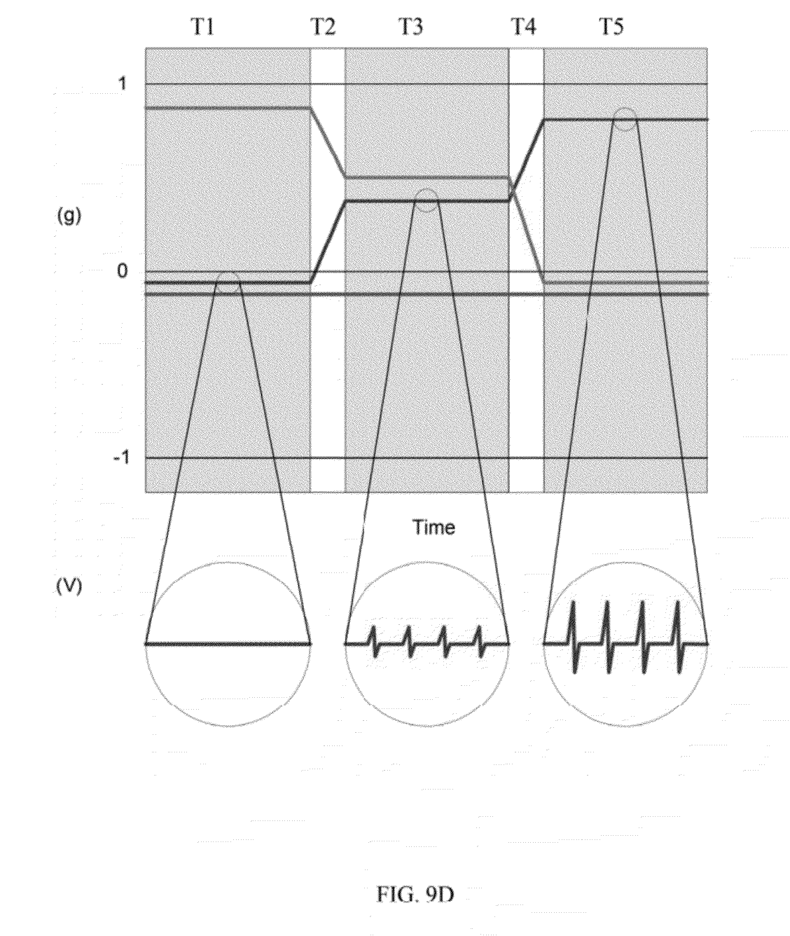

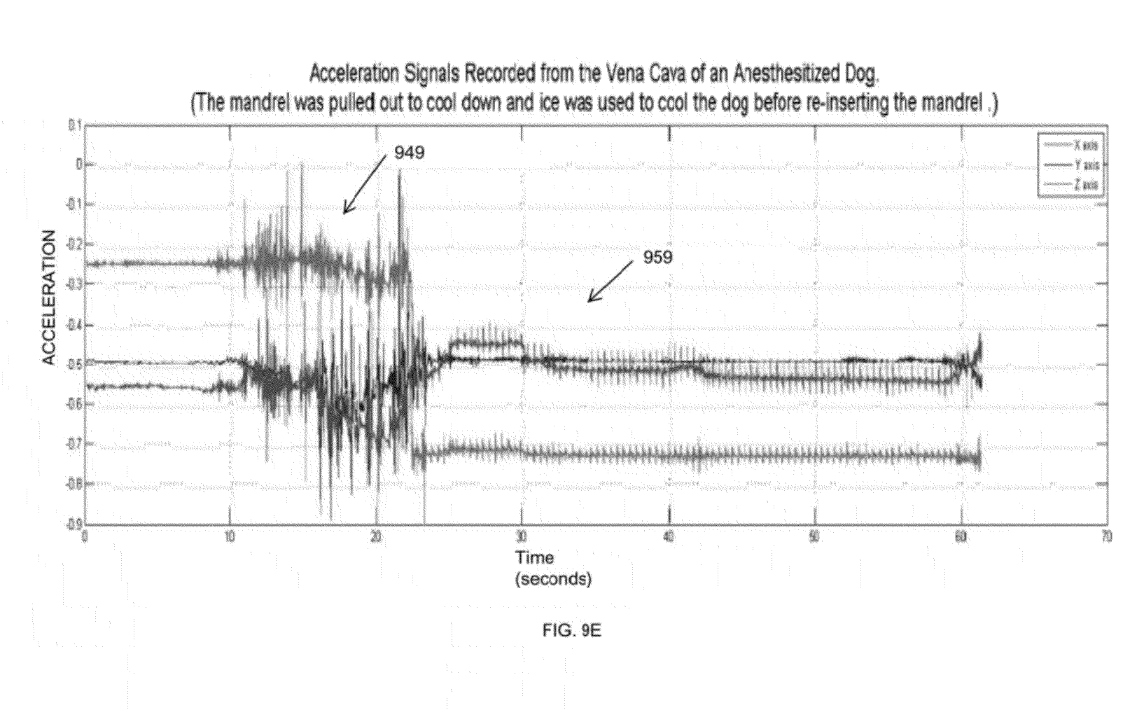

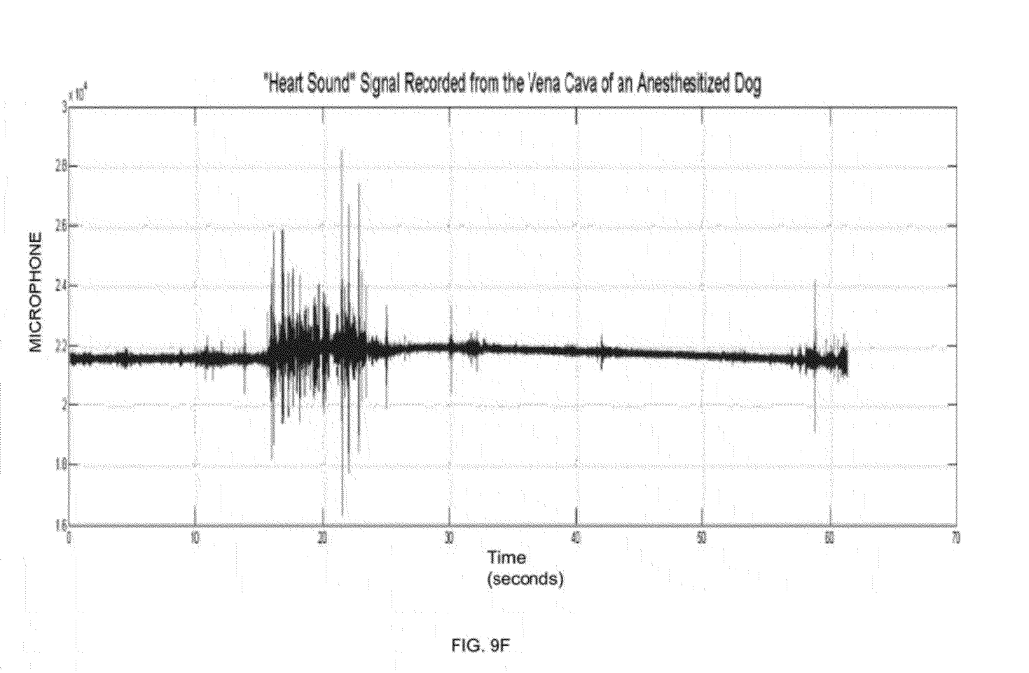

FIGS. 9A-9E illustrate various embodiments in which amplitudes of forces, motions, or sounds caused by cardiac functions such as the contraction and expansion of patient's heart 903 may be monitored and utilized to determine an IID dislodgement event according to various aspects of the invention described herein. As shown in FIG. 9A, a typical mammalian heart includes multiple chambers. These chambers contract or relax and thus cause blood to circulate through the vascular system. Blood enters and exits the heart via one or more valves that open and close in synchronization with cardiac contractions. Not only is blood caused to flow as a result of these contractions, but sound is produced as a result of the motion of cardiac chambers and the opening and closing of heart valves.

As shown in FIG. 9A, IID detector 906 may be adapted to detect the functions of heart 903. In one embodiment, IID detector 906 may be adapted to detect accelerations imparted on the IID (such as by monitoring movement of an unanchored portion of the IID, or by monitoring slight movement of an anchored portion of an IID) by the flow of blood caused by the functions of heart 903. In another embodiment, IID detector 906 may be adapted to detect sound caused by the functions of heart 903.

FIGS. 9B and 9C illustrate generally an IID 900 that includes dislodgement detector 906 adapted to detect the functions of heart 903 to determine a dislodgement event. As depicted in FIG. 9B, detector 906 is disposed at an initial position relatively far from heart 903 in vascular system 901. At this position, detector 906 may be adapted to detect sound or blood flow resulting from the functions of heart 903. Should IID 900 become dislodged from this initial position, detector 906 moves closer to the heart, as depicted in FIG. 9C. In this position, either accelerations imparted on detector 806 by the flow of blood, or heart sounds, will have greater amplitude because detector 806 is now much closer to heart 903. As such, detector 906 may be able to provide an indication of a dislodgement event.

A dislodgement detector 906 as depicted in FIGS. 9B and 9C may employ an accelerometer, a microphone, or both in order to detect the functions of heart to determine a dislodgement event. For example, a microphone may be deployed that is adapted to detect sounds of a particular frequency known to be associated with cardiac functions. In another example, an accelerometer may be adapted to detect such sounds. In an embodiment, an accelerometer may be adapted to detect the flow of blood resulting from cardiac functions.

FIGS. 9D-9F illustrate generally graphs depicting the results of experiments involving an IID that includes a dislodgement detector adapted to detect cardiac functions according to various aspects of the invention described herein. FIG. 9D illustrates an experiment similar to that depicted in FIG. 8F, except the dislodgement detector utilized in this experiment is adapted to, in addition to accelerations caused by the force of gravity, detect accelerations caused by the functions of the heart. As shown, during time period T1 when IID 900 is at an initial position further from heart 903, little or no acceleration caused by cardiac functions is detected by detector 906. During time period T3, however, detector 906 has become dislodged and has moved to a second position closer to heart 903. As depicted, accelerations caused by cardiac functions, such as movement imparted on detector 906 by the flow of blood exiting or entering heart 903, are detected. During time period T5, detector 906 has moved even closer to heart 903, and as such an amplitude of detected accelerations caused by cardiac function have increased.

FIGS. 9E-F depict results of similar experiments to the experiment shown in FIG. 9D, except in an initial position dislodgement detector is closer to heart 903, and a second position of detector 906 after dislodgement is further from heart 903. As shown, at 949 IID is anchored at an initial position, and dislodgement detector 906 is disposed at a position close to heart 903. As such, accelerations (or sounds) caused by cardiac functions have a relatively large amplitude. At 959, however, IID 900 has become dislodged and detector 906 has moved further from heart, and amplitudes of accelerations (or sounds) caused by heart functions have decreased substantially.

FIG. 9F depicts an embodiment in which detector 906 is adapted to detect sounds emitted by cardiac functions (via an accelerometer or microphone). As shown, at 979 IID is secured at a desired location, and at 989 IID has become dislodged. As a result, detector 906 has moved from an initial location close to heart 903 to a position further from heart 903. As depicted, the amplitude of heart sounds detected by detector 906 is much greater at the initial position in comparison to the dislodged position. As such, detector 906 my be able to provide a reliable indication of an IID dislodgement event.

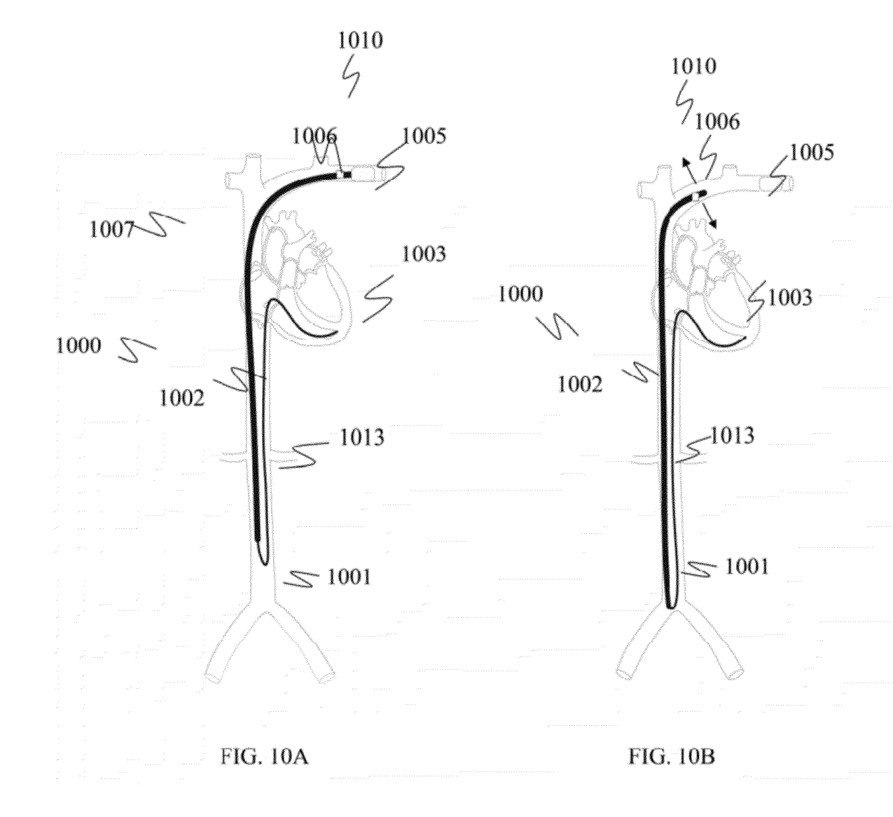

FIGS. 10A-B illustrate generally dislodgement 1006 detector adapted to detect whether detector 1006 is moving freely in the blood stream. As depicted in FIG. 10A, because IID 800 is secured in vessel 1001 by anchor 1005, atop end 1010 of IID main body 1002 is not able to move freely in vascular organ 1001. However, when IID 1000 has become dislodged from its initial position as depicted in FIG. 10B, the top end of IID main body may now be freely moving in vascular vessel 1001. To detect this movement, detector 1006 may be disposed near anchor 1005. Motion detected by detector 1006 may be monitored to determine whether a portion of IID 1000 expected to be anchored in vessel 1001 is freely moving. In one such embodiment, detector 1006 may be adapted to monitor whether detector 1006 is moving in directions parallel to vessel 1001. In an embodiment, detector 1006 may be adapted to compare indications of the movement of a portion of IID 1000 to one or more predetermined thresholds to determine a dislodgement event.

The embodiments of IID dislodgement detectors described herein may be employed standalone or in any combination to provide an indication of an IID dislodgement event. For example, an IID dislodgement detector that includes at least one accelerometer may be adapted to 1) determine g-vector accelerations imparted on the detector to determine an orientation or location of the detector, 2) monitor accelerations caused by the movement of an IID from a first position to a second position, 3) determine proximity to the heart by monitoring accelerations caused by cardiac functions (flow of blood, cardiac sounds), and/or 4) determine whether a portion of an IID is unexpectedly freely moving in a vascular organ. An IID may include any combination one or more accelerometer based dislodgement detectors and one or more separation detection dislodgement detectors as described above. Any combination of the above described embodiments is within the spirit and scope of the invention described herein.

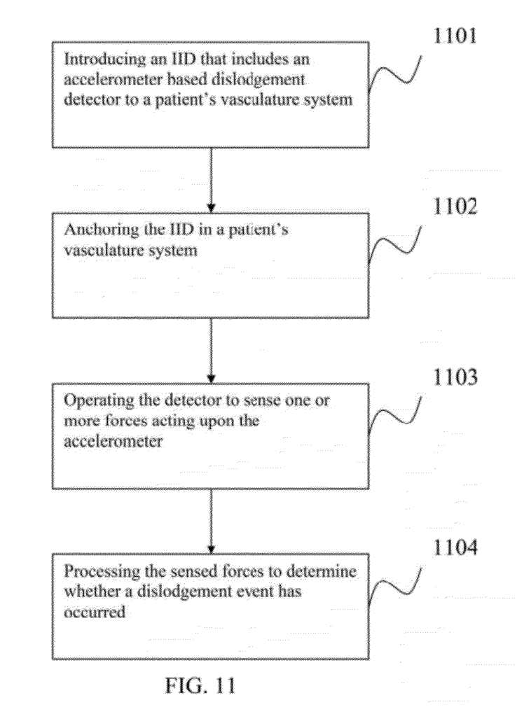

FIG. 11 illustrates generally a method of detecting dislodgement of an intravascular implantable medical device. At 1101, an IID that includes an accelerometer based dislodgement detector is introduced to patient's vascular system. At 1102, the IID is anchored in a patient's vascular system. In an embodiment, when the IID is known to be securely anchored in the vascular system, a calibration procedure is performed to establish one or more baseline conditions. At 1103, the detector is operated to sense one or more forces. In an embodiment, the one or more forces result from movement of the IID. In another embodiment, the one or more forces are caused by gravity. In still another embodiment, the one or more forces are imparted on the detector as a result of cardiac functions. In yet another embodiment, the one or more forces may indicate that a portion of the IID is freely moving in a vascular system. At 1104, the sensed forces are processed to determine whether a dislodgement event has occurred. In an embodiment, where the sensed forces indicate movement of the IID, such movement indicates that a dislodgement event has occurred. In another embodiment, where a baseline value of a gravitation vector has been established, the sensed forces are compared to the baseline value to determine whether a dislodgement event has occurred. In some embodiments, the sensed forces are compared to one or more predetermined thresholds or one or more previously sensed and stored indications of sensed forces to determine whether a dislodgement event has occurred.

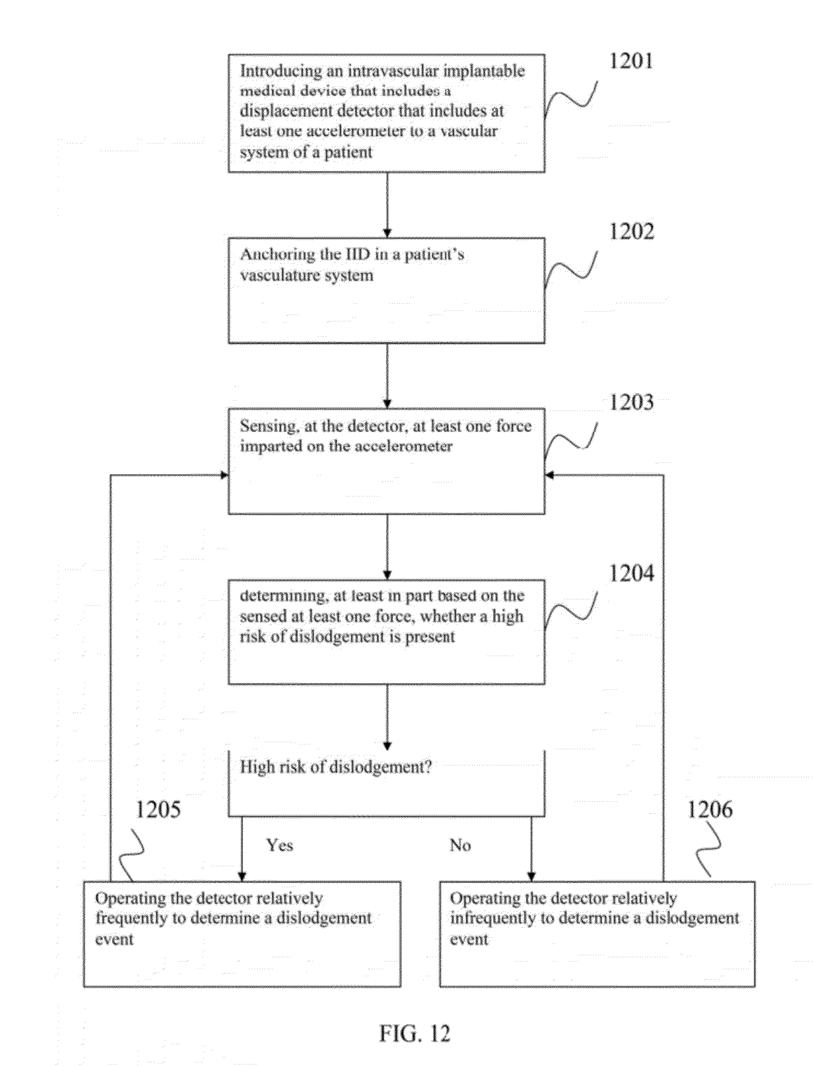

FIG. 12 is a flow chart diagram illustrating generally a method of detecting an IID dislodgement event according to various aspects of the invention described herein. At 1201, an intravascular implantable medical device that includes a dislodgement detector is introduced to a vascular system of a patient. In an embodiment, the device is calibrated at a time when it is known that the device is desirably anchored in the vascular system. At 1202, the device is anchored at a position in the vascular system. At 1203, the device is periodically operated to detect, via the accelerometer, at least one force imparted on the accelerometer. At 1204, the device is operated to determine whether a high risk of dislodgement is present. In an embodiment, the device is operated to determine a high risk of dislodgement based on the detected at least one force imparted on the accelerometer. At 1205, if is determined that a high risk of dislodgement exists, the device is operated relatively frequently to sense forces acting upon the accelerometer at 1203 to determine a dislodgement event. At 1206, if it is not determined that a high risk condition exists, the device is operated relatively infrequently to sense forces acting upon the accelerometer at 1203 to determine a dislodgement event.

In various additional embodiments, when the detector is operated at different frequencies at 1205 or 1206 to sense forces imparted on the accelerometer to determine a dislodgement event, the IID may also be adapted to determine whether a high risk of dislodgement is present. As such, IID may be adapted to continually monitor and vary a rate of detection based on whether or not a risk of dislodgement is present.

In a similar embodiment to that depicted in FIG. 12, the IID may be operated to modify a rate of dislodgement event detection according to a risk profile. According to this embodiment, instead of merely “high risk” and “low risk” conditions, the IID may be operable to detect a variety of different forces and other conditions that may alone or in combination allow the detector to determine a level of risk. As such, in an embodiment, the IID may be adapted to determine a risk level and adjust how frequently the detector attempts to determine a dislodgement event.

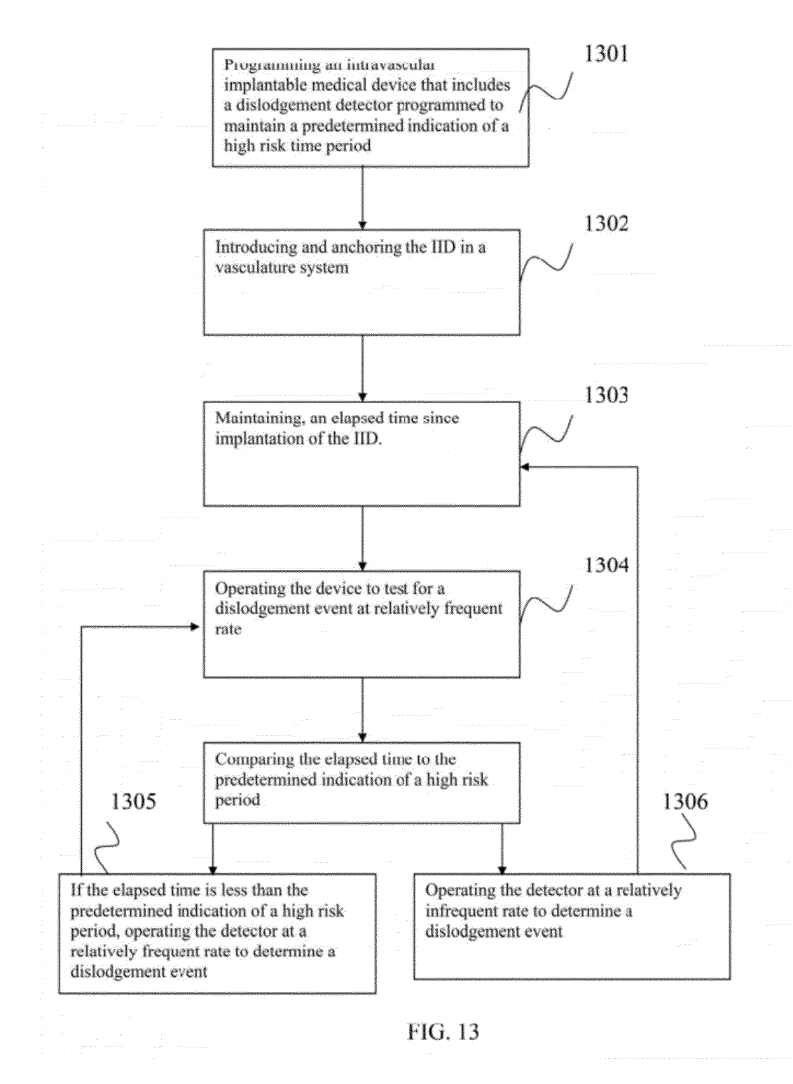

FIG. 13 is a flow chart diagram illustrating generally a method of operating an IID dislodgement detector to operate at differing sample rates based on a high risk time period according to various aspects of the invention described herein. As illustrated in FIG. 13, at 1301 an intravascular implantable medical device that includes a dislodgement detector is programmed to maintain a predetermined indication of a high risk time period. At 1302, the device is introduced to and anchored in a patient's vascular system. At 1303, the device is operated to maintain an indication of the time elapsed since the device was implanted. At 1304, the device is operated to test for a dislodgement event at relatively frequent rate. At 1305, the indication of elapsed time is compared to the predetermined indication of high risk time period. At 1306, if the operating time period is less than the initial time period, the device continues to test for a dislodgement event at the relatively frequent rate. At 1307, if the operating time period is greater than the initial time period, the device is operated to test for a dislodgement event at a less frequent rate.

The embodiment depicted in FIG. 13 improves IID safety, because an IID is more likely to become dislodged from an anchored position during an initial time period subsequent to implantation in a vascular system as discussed herein. By operating the IID as described above, safety is improved while minimizing drain on batteries of an IID.

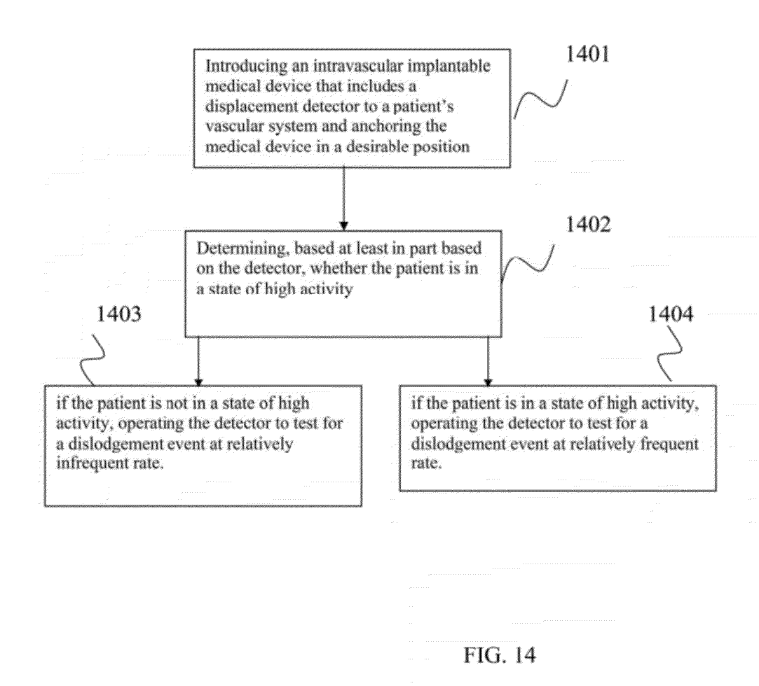

FIG. 14 is a flow chart diagram illustrating generally a method of operating a dislodgement detector based on a patient's physical activity according to various aspects of the invention described herein. At 1401 an intravascular implantable medical device is introduced to and anchored in a patient's vascular system in a desirable position. In an embodiment, the dislodgement detector is calibrated when the device is anchored in a known position. At 1402, the device is adapted to determine, at least in part based on an accelerometer included in the dislodgement detector, whether the patient is in a state of high activity. At 1403, if the patient is not in a state of high activity, the device is operated to test for a dislodgement event at relatively infrequent rate. At 1404, if the patient is in a state of high activity, the device is operated to test for a dislodgement event at a relatively frequent rate. In an embodiment, the device is operated to test for dislodgement at a relatively frequent rate for a predetermined time period after a high activity state has been detected.

The embodiment depicted in FIG. 14 improves IID safety, because an IID is more likely to become dislodged from an anchored position during periods of physical activity of the patient. By operating the IID as described above, safety is improved while minimizing drain on batteries of an IID.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. An intravascular implantable medical device, comprising:
an implant engageable at an implant position within a living body; and
a dislodgement detector positioned to detect dislodgement of the implant from the implant position.

2. The medical device of claim 1, wherein the implant includes a main body and an anchor adapted to engage a blood vessel to secure the main body in a vasculature of a patient, wherein the dislodgement detector includes at least one separation detection circuit adapted to provide an coupling between the main body and the anchor, the separation detection circuit arranged such that if the main body becomes separated from the anchor, a detectable coupling between the main body and the anchor is severed, thus providing an indication of a dislodgement event.

3. The medical device of claim 2, wherein the coupling is an electrical, optical, or magnetic coupling.

4. The medical device of claim 3, wherein the separation detection circuit is an electrical conductor constructed to form a current loop between the main body and the anchor.

5. The medical device of claim 4, wherein the electrical conductor is adapted to break upon separation of the main body from the anchor.

6. The medical device of claim 4, wherein the main body includes electrical circuitry adapted to cause a current to flow through the electrical conductor, and to monitor current flow through the electrical conductor, said electrical circuitry adapted to provide an indication of a dislodgement event upon detection of a discontinuation of current flow through the electrical conductor.

7. The medical device of claim 1, wherein the dislodgement detector includes at least one accelerometer adapted to detect forces and/or movement.

8. The medical device of claim 7, wherein the accelerometer is adapted to detect a magnitude of g-vector forces along a vertical axis.

9. The medical device of claim 8, wherein the detector is adapted to compare an established baseline g-vector amplitude and a current g-vector amplitude to determine a dislodgement event.

10. The medical device of claim 9, wherein the established baseline g-vector amplitude is a g-vector amplitude determined when the patient is in an upright, resting position.

11. The medical device of claim 7, wherein the detector comprises a multi-axis accelerometer adapted to detect g-vector accelerations along a plurality of dimensional axes.

12. The medical device of claim 1, wherein the detector comprises a microphone adapted to detect one or more sounds generated by the heart, for monitoring a proximity of the detector to the heart and detecting a change in proximity of the detector to the heart.

13. A method of detecting dislodgment of an intravascular medical device, comprising:
introducing an intravascular implantable medical device having a dislodgment detector into a vasculature of a patient, the dislodgement detector including an accelerometer;
anchoring the medical device in the vasculature;
operating the detector to sense one or more forces acting on the accelerometer; and
processing the sensed forces to determine whether a dislodgement event has occurred.

14. A method of detecting dislodgment of an intravascular medical device, comprising:
introducing an intravascular implantable medical device having a dislodgment detector into a vasculature of a patient;
anchoring the medical device in the vasculature, wherein the dislodgement detector includes at least one separation detection circuit adapted to provide an electrical, optical or magnetic coupling between the main body and the anchor;
operating the detector to sense interruption of the coupling between the main body and the anchor to determine whether a dislodgement event has occurred.
